(19) United States
(12) Patent Application Publication
(10) Pub. No.: US 2026/0177538 A1
(43) Pub. Date: Jun. 25, 2026

(54) METHOD FOR DETERMINING OPTIMAL CONSTRUCTION TEMPERATURE IN MODIFIED ASPHALT AT DIFFERENT CONTENTS OF MODIFIER

(71) Applicant: Changsha University of Science and Technology, Changsha (CN)

(72) Inventors: Bo LIANG, Changsha (CN); Zheng LIU, Changsha (CN); Wei LIAO, Changsha (CN); Jianlong ZHENG, Changsha (CN)

(21) Appl. No.: 19/417,403

(22) Filed: Dec. 12, 2025

Related U.S. Application Data
(63) Continuation of application No. PCT/CN2025/135400, filed on Nov. 17, 2025.

(30) Foreign Application Priority Data
Dec. 19, 2024 (CN) 2024118771820

(51) Int. Cl. G01N 33/42; G01N 11/00; G01N 11/14

(52) U.S. Cl. CPC G01N 33/42; G01N 11/14; G01N 2011/0093

(57) ABSTRACT

A method for determining an optimal construction temperature of modified asphalt at different contents of a modifier including: S1, measuring a viscosity of modified asphalt samples at a temperature; S2, at temperature T₁, plotting a curve with a modifier content as x-coordinate and the viscosity of the modified asphalt samples as y-coordinate; determining a transition point O_{T1}; S3, taking the temperature T₁ as the optimal construction temperature of the modified asphalt corresponding to a specific modifier content; S4, establishing a relationship between the modifier content and the optimal construction temperature, and determining the optimal construction temperature of the modified asphalt at that content based on an actual modifier content of the modified asphalt during construction. The method can establish the correspondence between the content of a specific modifier and the optimal construction temperature and build a database of optimal construction temperatures for modified asphalt containing different modifiers.

### CROSS-REFERENCE TO RELATED APPLICATION

This application claims priority to Chinese patent application No. CN202411877182.0, filed to China National Intellectual Property Administration (CNIPA) on Dec. 19, 2024, which is herein incorporated by reference in its entirety.

### TECHNICAL FIELD

The disclosure relates to the field of pavement construction technologies, and more particularly to a method for determining an optimal construction temperature in modified asphalt.

### BACKGROUND

With the rapid advancement of road construction technologies, particularly to polymer modification techniques, have significantly improved the pavement performance, including its high-temperature stability, low-temperature cracking resistance, and durability. Specifically, the thermoplastic polymer styrene-butadiene-styrene (SBS) is the most successfully applied polymer modifier. SBS-modified asphalt exhibits excellent high-temperature rutting resistance and good low-temperature crack resistance, making it one of the primary materials currently used in asphalt pavement construction. Compared with matrix asphalt, the SBS-modified asphalt demonstrates considerable advantages in terms of physical properties, storage stability, and cost-effectiveness. In practical applications, the construction temperature of modified asphalt affects the extent of asphalt aging. Selecting an appropriate application temperature can save energy, reduce carbon emissions, and simultaneously ensure favorable pavement performance. However, in the related art and conventional construction practices, the construction temperature is often chosen on-site based on empirical judgment, without differentiation among modified asphalts containing different types of modifiers. The determination of construction temperature lacks a consistent, effective standard and is subject to various human factors, making it difficult to fully realize the optimal performance potential of modified asphalt.

### SUMMARY

In order to overcome problems in the related art, the disclosure provides a method for determining an optimal construction temperature of modified asphalt. At various temperatures, viscosity variation curves of modified asphalts with different contents of a modifier are established. By performing a first derivative on these curves, a transition point O where viscosity changes with the content of the modifier is obtained. An x-coordinate value corresponding to the transition point O represents an upper limit of the content of the modifier in the modified asphalt at that construction temperature, and a reasonable construction temperature of the modified asphalt at this content is the temperature corresponding to the viscosity at that moment.

To solve the aforementioned technical problems, technical solution provided by the disclosure is as follows.

Specifically, a method for determining an optimal construction temperature of modified asphalt with different contents of a modifier includes the following steps:
S1: preparing modified asphalt samples with the different contents of the modifier and measuring a viscosity of the modified asphalt samples at different temperatures Tₙ, where n is a positive integer;
S2: at temperature T₁, plotting a curve with a modifier content as x-coordinate and the viscosity of the modified asphalt samples as y-coordinate; determining a transition point O_{T1} on the curve where the viscosity changes abruptly with the modifier content;
S3: taking an x-coordinate value corresponding to the transition point O_{T} as a modifier content C_{T1}, taking the temperature T₁ as the optimal construction temperature of the modified asphalt corresponding to the modifier content C_{T}, of the transition point O_{T1}; and
S4: obtaining transition points O_{Tn} at the different temperatures Tₙ through testing, establishing relationships between modifier contents C_{Tn} and optimal construction temperatures Tₙ, and determining the optimal construction temperature of the modified asphalt at a specific content of the modified asphalt during actual construction.

The design concept of the above technical solution lies in using viscosity, which measures the resistance of fluid flow, to characterize the friction resistance between molecules when fluids flow at different temperatures. Investigating viscosity indicators of asphalt in its flowing state can determine the mixing and compaction temperatures of various asphalt mixtures. The Strategic Highway Research Program (SHRP) in the United States uses Brookfield viscometers to test viscosity values of asphalt, and China has also incorporated the Brookfield viscosity testing method into its “Standard Test Methods for Bitumen and Bituminous Mixtures for Highway Engineering” (JTG E20-2019). The disclosure establishes viscosity variation curves of modified asphalts with different modifier contents. When the modifier content exceeds a certain value, the viscosity of the modified asphalt sharply increases, defining this point as the transition point (performing linear fitting on the trend of slope changes, the point where the slope increases abruptly is identified as the transition point). The modifier content corresponding to the transition point is key to balancing the high-temperature performance and workability of asphalt during construction. The inventor found that when the modifier content exceeds the value corresponding to the transition point, the sharp increase in asphalt viscosity significantly increases the difficulty and cost of construction, making it reasonable to choose this temperature as the construction temperature within this range.

In an embodiment, the transition point O_{T1} in the step S2 and the transition points O_{Tn} in the step S3 are determined by the following method:
performing a first derivative on the curve to obtain a derivative curve of viscosity versus content; when a slope of the derivative curve undergoes an abrupt change as the modifier content increases, the modifier content corresponding the abrupt change is recorded as the transition point.

In an embodiment, in the step S1, the contents of the modifier vary linearly in a gradient between 1% and 10%.

In an embodiment, in the step S1, a measurement temperature range for the modified asphalt samples is 80° C. to 210° C.

In an embodiment, in the step S1, a torque range of a viscometer is controlled between 10% and 98% during viscosity measurement.

In an embodiment, the modifier is styrene-butadiene-styrene or a mixture containing the styrene-butadiene-styrene.

In an embodiment, the method further includes testing a relationship between the modifier content and the optimal construction temperature for modified asphalt with different modifier types according to the step S1 to the step S4, and constructing an optimal construction temperature database for different modifiers; determining the optimal construction temperature of the modified asphalt based on a specific modifier type and a specific modifier content of the modified asphalt during the actual construction.

In an embodiment, when the transition point cannot be determined based on the curve in the step S2, a viscosity measurement temperature for the modified asphalt samples is lowered until the transition point is determined.

Compared with the related art, beneficial effects of the disclosure are as follows.

The disclosure provides the method for determining the optimal construction temperature of the modified asphalt at the different contents of the modifier. By using this method, a correlation between the modifier content and the optimal construction temperature of modified asphalt can be established, and a database of optimal construction temperatures for modified asphalts containing various modifiers can also be constructed. In actual production processes, this enables construction parties to determine the optimal construction temperature based on the type of modifier and the actual modifier content, thereby eliminating the influence of human factors and uncertain information present in current methods for determining construction temperature. Construction at the optimal construction temperature facilitates easier placement and compaction of the modified asphalt material, improves construction efficiency and shortens project duration, while ensuring good adhesion and density of the asphalt mixture, thus enhancing the load-bearing capacity and durability of the pavement. Reasonably determining the asphalt construction temperature not only improves construction quality and efficiency but also reduces long-term maintenance costs and environmental impact.

### BRIEF DESCRIPTION OF DRAWINGS

In order to more clearly illustrate technical solutions in embodiments of the disclosure or in the related art, the accompanying drawings required for describing the embodiments or the related art will be briefly introduced below. Apparently, the drawings described below are only some embodiments of the disclosure, for those skilled in the art, other drawings may also be obtained based on these drawings without making creative efforts.

FIG. 1 illustrates a first-derivative plot showing variation of viscosity of modified asphalt with modifier content at 135° C. according to embodiment 1 of the disclosure.

FIG. 2 illustrates a first-derivative plot showing variation of the viscosity of the modified asphalt with the modifier content at 175° C. according to the embodiment 1 of the disclosure.

FIG. 3 illustrates a first-derivative plot showing variation of the viscosity of the modified asphalt with the modifier content at 190° C. according to the embodiment 1 of the disclosure.

FIG. 4 illustrates a first-derivative plot showing variation of the viscosity of the modified asphalt with the modifier content at 205° C. according to the embodiment 1 of the disclosure.

FIG. 5 illustrates a first-derivative plot showing variation of viscosity of modified asphalt with modifier content at 135° C. according to embodiment 2 of the disclosure.

FIG. 6 illustrates a first-derivative plot showing variation of the viscosity of the modified asphalt with the modifier content at 175° C. according to the embodiment 2 of the disclosure.

FIG. 7 illustrates a first-derivative plot showing variation of the viscosity of the modified asphalt with the modifier content at 190° C. according to the embodiment 2 of the disclosure.

FIG. 8 illustrates a first-derivative plot showing variation of the viscosity of the modified asphalt with the modifier content at 205° C. according to the embodiment 2 of the disclosure.

FIG. 9 illustrates a first-derivative plot showing variation of viscosity of modified asphalt with modifier content at 135° C. according to embodiment 3 of the disclosure.

FIG. 10 illustrates a first-derivative plot showing variation of the viscosity of the modified asphalt with the modifier content at 175° C. according to the embodiment 3 of the disclosure.

FIG. 11 illustrates a first-derivative plot showing variation of the viscosity of the modified asphalt with the modifier content at 190° C. according to the embodiment 3 of the disclosure.

### DETAILED DESCRIPTION OF EMBODIMENTS

To facilitate understanding of the disclosure, the following text will provide a more comprehensive and detailed description of the disclosure with reference to the accompanying drawings and illustrated embodiments. However, the scope of protection of the disclosure is not limited to the specific embodiments described herein.

Unless otherwise defined, all professional terms used hereinafter have the same meanings as commonly understood by those skilled in the art. The professional terms used herein are solely for the purpose of describing specific embodiments and are not intended to limit the scope of protection of the disclosure.

Unless specifically stated otherwise, all kinds of raw materials, reagents, instruments, and equipment used in the disclosure can be purchased on the market or prepared by existing methods.

### Embodiment 1

A method for determining an optimal construction temperature of modified asphalt at different contents of a modifier according to this embodiment includes the following steps.
(1) Using matrix asphalt 1 (YH-79, from SINOPEC BALING PETROCHEMICAL CO., LTD., Yueyang, Hunan, China), a certain amount of matrix asphalt is weighed and heated to 180° C. Linear SBS modifiers are added at a shear rate of 700 revolutions per minute abbreviated as r/min (addition amounts being 0%, 1%, 2%, 3%, 4%, 5%, 6%, 7%, 8%, 9% respectively). The temperature is maintained at 180° C. Then, the shear rate is adjusted to 7000 r/min while keeping the temperature constant, and shearing is performed for 2 hours to obtain sheared samples. The sheared samples are then transferred to a disperser and dispersed at 700 r/min for 2 hours to prepare a series of modified asphalt samples. The prepared modified asphalt samples are controlled using the single-variable method, ensuring that the only variable among all samples is the content of SBS modifier.
(2) Viscosity tests are performed on the modified asphalt samples at various temperatures ranging from 80° C. to 210° C. (in this embodiment, 135° C., 175° C., 190° C., and 205° C.). According to the specific test temperature and the specific type of asphalt sample, an appropriate rotor model is chosen, and torque reading is controlled within the range of 10%-98%. Some of the test data are shown in Table 1 below.

TABLE 1
Relationship between SBS content in SBS-modified
asphalt and viscosity of SBS-modified asphalt

Viscosity (Pa · s)

| SBS content (%) | 135° C. | 175° C. | 190° C. | 205° C. |
|---|---|---|---|---|
| 0 | 0.50 | 0.09 | 0.07 | 0.04 |
| 1 | 0.89 | 0.17 | 0.09 | 0.06 |
| 2 | 1.11 | 0.19 | 0.13 | 0.07 |
| 3 | 1.44 | 0.28 | 0.17 | 0.10 |
| 4 | 1.75 | 0.39 | 0.25 | 0.13 |
| 5 | 2.42 | 0.47 | 0.34 | 0.19 |
| 6 | 2.95 | 0.74 | 0.56 | 0.37 |
| 7 | 4.67 | 0.81 | 0.75 | 0.54 |
| 8 | 11.03 | 3.70 | 1.07 | 0.92 |
| 9 | 16.30 | 7.98 | 2.03 | 1.32 |

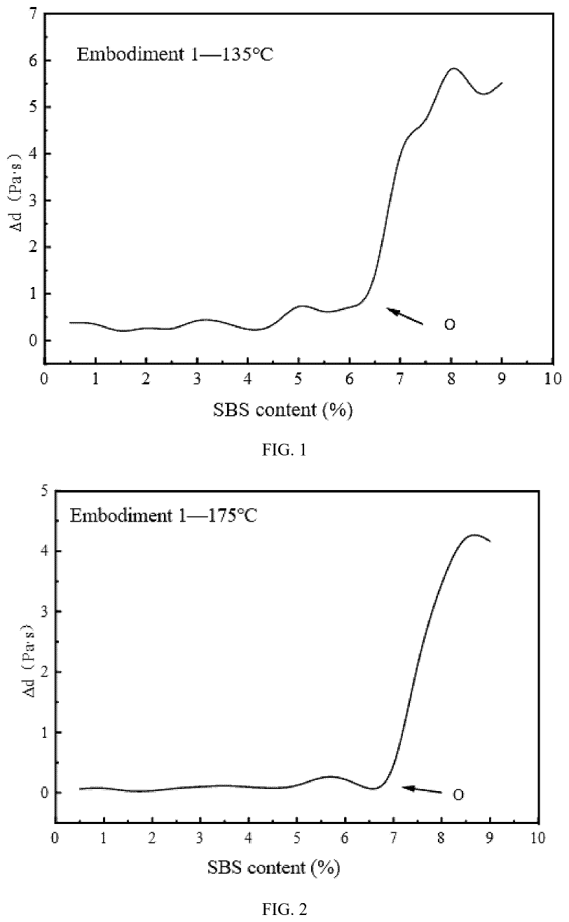

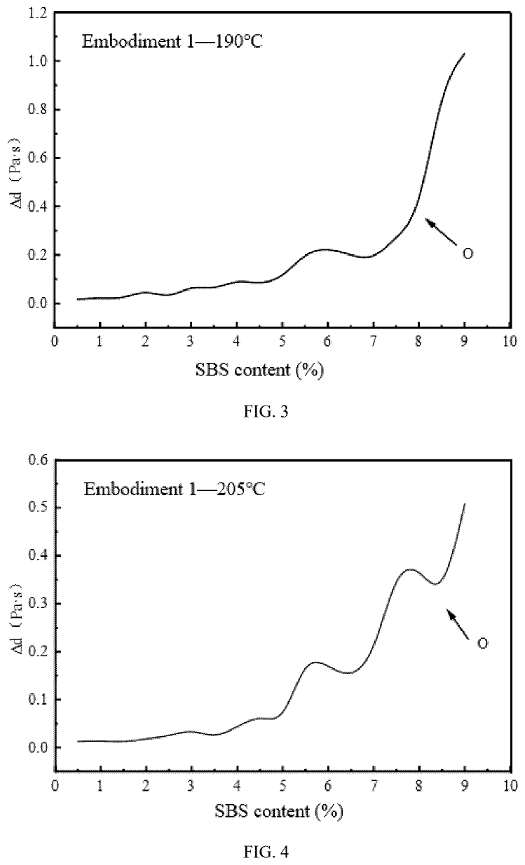

(3) At a specific temperature, a curve (i.e., viscosity curve) is plotted with modifier content as the x-coordinate and the viscosity of the modified asphalt sample as the y-coordinate. By performing a first derivative on the viscosity curve, the transition point O where the viscosity of the modified asphalt exhibits an abrupt change with respect to the modifier content is identified. By taking the derivative of the viscosity curve and analyzing the viscosity derivative curve, for ease of understanding, the derivative of viscosity with respect to SBS content was denoted as Δd=Δη/ΔSBS content. Thus, curves showing the variation of viscosity derivative with SBS content at different temperatures are obtained. By performing a first-derivative on these curves, the transition points at 135° C., 175° C., 190° C., and 205° C. are identified, with the corresponding SBS contents being 6.5%, 7.0%, 8.0%, and 8.5%, respectively. The first-derivative plots of modified asphalt viscosity versus modifier content at 135° C., 175° C., 190° C., and 205° C. are shown in FIGS. 1-4, indicating that the corresponding temperature can be used as the optimal construction temperature for asphalt when the modifier content reaches the value associated with each transition point. Additionally, at 135° C., a minor transition point appears in the viscosity derivative curve at approximately 5% SBS content, which corresponds to the phase transition point of SBS in asphalt. Compared with the aforementioned transition point, this point exhibits a less pronounced change and is therefore relatively easy to distinguish.
(4) Based on the steps (1)-(3), the relationship between SBS modifier content and optimal construction temperature is established, specifically, by taking the smallest SBS content among all transition points as the starting threshold. When the modifier content is less than or equal to 6.5%, the corresponding temperature of 135° C. is selected as the optimal construction temperature; when the modifier content is greater than 6.5% and less than or equal to 7.0%, the corresponding temperature of 175° C. is selected as the optimal construction temperature; when modifier content is greater than 7.0% and less than or equal to 8.0%, the corresponding temperature of 190° C. is selected as the optimal construction temperature; and when the modifier content is greater than 8.0% and less than or equal to 8.5%, the corresponding temperature of 205° C. is selected as the optimal construction temperature. In actual production, depending on the required precision, the test temperatures in the step (2) can be further refined (e.g., testing every 2° C. across the range of 80° C.-210° C.) to obtain more precise optimal construction temperatures for different modifier contents. Considering space limitations and practical needs, this embodiment only selects four temperatures for testing, which is sufficient to determine appropriate construction temperatures over certain modifier content ranges. Further refinement of test temperatures can yield even more accurate optimal construction temperatures.

### Embodiment 2

A method for determining an optimal construction temperature of modified asphalt at different contents of a modifier in this embodiment includes the following steps.
(1) The preparation method for the modified asphalt samples in this embodiment is essentially consistent with that in the embodiment 1, with the only difference being that linear SBS modifier and 0.15% sulfur are added to the matrix asphalt.
(2) Viscosity tests are performed on the modified asphalt samples at different temperatures, with the test temperature range set from 80° C. to 210° C. According to the specific test temperature and the specific type of asphalt sample, an appropriate rotor model is chosen, and torque reading is controlled within the range of 10%-98%. Some of the test data are shown in Table 2 below.

TABLE 2
Relationship between SBS content in S/SBS-modified
asphalt and viscosity of S/SBS-modified asphalt

Viscosity (Pa · s)

| SBS content (%) | 135° C. | 175° C. | 190° C. | 205° C. |
|---|---|---|---|---|
| 0 | 0.50 | 0.09 | 0.07 | 0.04 |
| 1 | 1.21 | 0.24 | 0.11 | 0.08 |
| 2 | 1.64 | 0.35 | 0.17 | 0.13 |
| 3 | 2.35 | 0.51 | 0.29 | 0.17 |
| 4 | 4.42 | 0.73 | 0.42 | 0.22 |
| 5 | 6.95 | 0.94 | 0.74 | 0.49 |
| 6 | 9.67 | 1.56 | 0.88 | 0.63 |
| 7 | 14.03 | 6.40 | 1.21 | 0.82 |
| 8 | 19.22 | 10.31 | 1.51 | 1.18 |

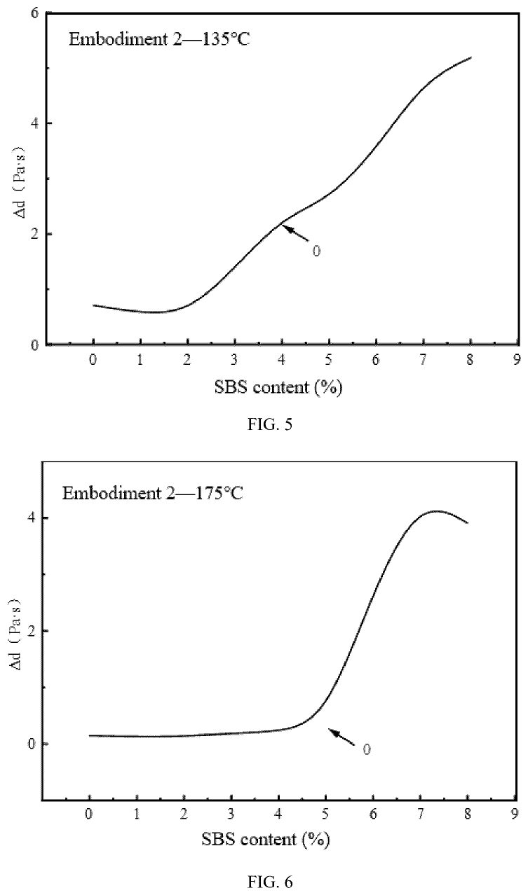

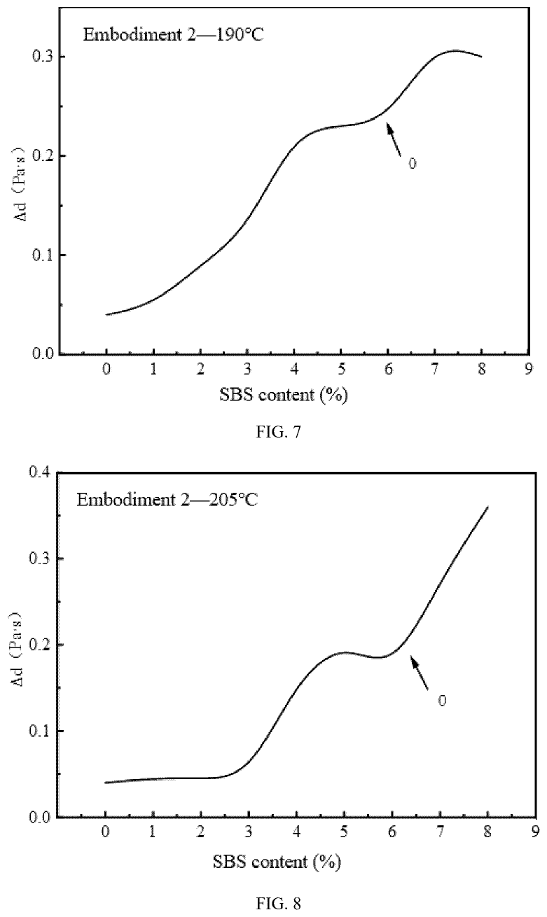

(3) At a specific temperature, a curve (i.e., viscosity curve) is plotted with modifier content as the x-coordinate and the viscosity of the modified asphalt sample as the y-coordinate. By performing a first derivative on the viscosity curve, the transition point O where the viscosity of the modified asphalt exhibits an abrupt change with respect to the modifier content is identified. By taking the derivative of the viscosity curve and analyzing the viscosity derivative curve, for ease of understanding, the derivative of viscosity with respect to SBS content was denoted as Δd=Δη/ΔSBS content. Thus, curves showing the variation of viscosity derivative with SBS content at different temperatures are obtained. By performing a first-derivative on these curves, the transition points at 135° C., 175° C., 190° C., and 205° C. are identified, with the corresponding SBS contents being 4.0%, 5.0%, 6.0%, and 6.5%, respectively. The first-derivative plots of modified asphalt viscosity versus modifier content at 135° C., 175° C., 190° C., and 205° C. are shown in FIGS. 5-8, indicating that the corresponding temperature can be used as the optimal construction temperature for asphalt when the modifier content reaches the value associated with each transition point. Additionally, at 135° C., a minor transition point O also appears in the viscosity derivative curve at approximately 5% SBS content, which corresponds to the phase transition point of SBS in asphalt.
(4) Based on the steps (1)-(3), the relationship between SBS modifier content and optimal construction temperature is established, specifically, by taking the smallest SBS content among all transition points as the starting threshold. When the modifier content is less than or equal to 4.0%, the corresponding temperature of 135° C. is selected as the optimal construction temperature; when modifier content is greater than 4.0% and less than or equal to 5.0%, the corresponding temperature of 175° C. is selected as the optimal construction temperature; when modifier content is greater than 5.0% and less than or equal to 6.0%, the corresponding temperature of 190° C. is selected as the optimal construction temperature; and when modifier content is greater than 6.0% and less than or equal to 6.5%, the corresponding temperature of 205° C. is selected as the optimal construction temperature.

When sulfur is added to SBS-modified asphalt, SBS undergoes a crosslinking reaction with sulfur during the formation of its network structure, resulting in a vulcanized SBS network structure. This reduces the temperature sensitivity of SBS-modified asphalt and enhances its high-temperature performance and compatibility. During modification, polystyrene segments in SBS are surrounded by polybutadiene segments, with polystyrene and polybutadiene interconnected to form a network structure. Upon addition of sulfur, the polybutadiene blocks in SBS generate chemically crosslinked domains and form more complex macromolecules. The resulting crosslinked network structure arises from the combined effects of physical and chemical interactions, and the sulfur-induced crosslinking creates a more stable structure. The chemical reaction between sulfur and SBS-modified asphalt affects the performance of the modified asphalt, causing the viscosity transition point of the modified asphalt to appear earlier by approximately 0%-2%. However, this does not alter the overall trends of viscosity variation or fluorescence behavior. Therefore, the method described in this embodiment is also applicable to polymer-modified asphalts containing stabilizers such as sulfur, demonstrating the broad applicability of the disclosure to various types of modifiers.

### Embodiment 3

A method for determining an optimal construction temperature of modified asphalt at different contents of a modifier in this embodiment includes the following steps.
(1) The preparation method for the modified asphalt samples in this embodiment is essentially consistent with that in the previous embodiments, with the only difference being that star-shaped SBS modifier and 3% nano-organo-montmorillonite (OMMT) are added to the matrix asphalt.
(2) Viscosity tests are performed on the modified asphalt samples at different temperatures, with the test temperature range set from 80° C. to 210° C. According to the specific test temperature and the specific type of asphalt sample, an appropriate rotor model is chosen, and torque reading is controlled within the range of 10%-98%. Some of the test data are shown in Table 3 below.

TABLE 3
Relationship between SBS content in OMMT/SBS-modified
asphalt and viscosity of OMMT/SBS-modified asphalt

| | Viscosity (Pa · s) | | |
|---|---|---|---|
| SBS content (%) | 135° C. | 175° C. | 190° C. |
| 0 | 0.50 | 0.09 | 0.07 |
| 2 | 1.11 | 0.19 | 0.09 |
| 3 | 1.44 | 0.28 | 0.14 |
| 4 | 1.75 | 0.39 | 0.19 |
| 5 | 2.42 | 0.47 | 0.28 |
| 6 | 2.95 | 0.74 | 0.39 |
| 7 | 4.67 | 0.81 | 0.61 |
| 8 | 11.03 | 3.70 | 0.80 |

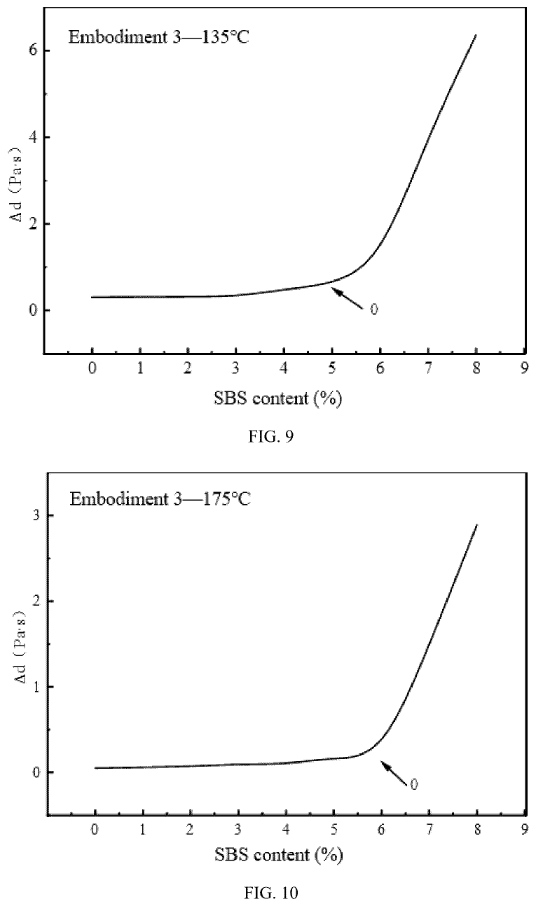

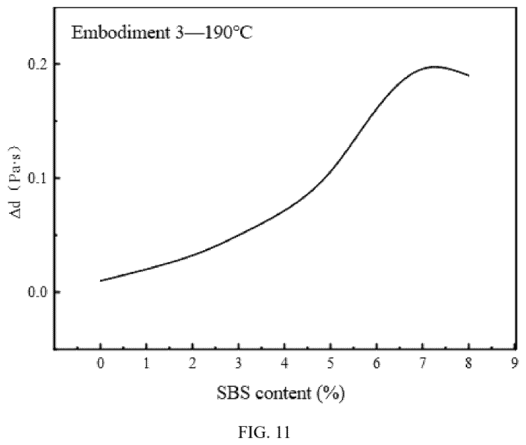

(3) A curve (i.e., viscosity curve) is plotted with modifier content as the x-coordinate and the viscosity of the modified asphalt sample as the y-coordinate. By performing a first derivative on the viscosity curve, the transition point O where the viscosity of the modified asphalt exhibits an abrupt change with respect to the modifier content is identified (in this embodiment, when testing at 190° C. and 205° C., the Δd transition point is found to be no longer obvious, indicating that the construction temperatures for the asphalt are all below 190° C.; therefore, only the curves at 135° C. and 175° C. are analyzed). By taking the derivative of the viscosity curve and analyzing the viscosity derivative curve, curves showing the variation of viscosity derivative with SBS content at different temperatures are obtained, as illustrated in FIGS. 9-11 for 135° C., 175° C., and 190° C., respectively. Based on the first derivatives of these curves, the transition points O at 135° C. and 175° C. are identified, with the corresponding SBS contents being 5.0% and 6.0%, respectively. This indicates that, when the modifier content does not exceed the value corresponding to transition point O, the respective temperature can be used as the construction temperature for the asphalt.
(4) Based on the steps (1)-(3), the relationship between SBS modifier content and optimal construction temperature is established, specifically, by taking the smallest SBS content among all transition points as the starting threshold. When the modifier content is less than or equal to 5.0%, the corresponding temperature of 135° C. is selected as the optimal construction temperature; and when modifier content is greater than 5.0% and less than or equal to 6.0%, the corresponding temperature of 175° C. is selected as the optimal construction temperature.

The above description provides a further detailed explanation of the disclosure in conjunction with specific embodiments and should not be construed as limiting the scope of the disclosure solely to these embodiments. For those skilled in the art to which the disclosure pertains, various simple modifications or substitutions may be made without departing from the inventive concept, and all such modifications and substitutions shall be deemed within the scope of protection of the disclosure.

What is claimed is:

1. A method for determining an optimal construction temperature of modified asphalt at different contents of a modifier, comprising the following steps:
S1: preparing modified asphalt samples with the different contents of the modifier and measuring a viscosity of the modified asphalt samples at different temperatures Tₙ, where n is a positive integer;
S2: at temperature T₁, plotting a curve with a modifier content as x-coordinate and the viscosity of the modified asphalt samples as y-coordinate; determining a transition point O_{T1} on the curve where the viscosity changes abruptly with the modifier content;
S3: taking an x-coordinate value corresponding to the transition point O_{T1} as a modifier content C_{T1}, taking the temperature T₁ as the optimal construction temperature of the modified asphalt corresponding to the modifier content C_{T1} of the transition point O_{T1}; and
S4: obtaining transition points O_{Tn} at the different temperatures Tₙ through testing, establishing relationships between modifier contents C_{Tn} and optimal construction temperatures Tₙ, and determining the optimal construction temperature of the modified asphalt at a specific content of the modified asphalt during actual construction.

2. The method for determining the optimal construction temperature of the modified asphalt at the different contents of the modifier as claimed in claim 1, wherein the transition point O_{T1} in the step S2 and the transition points O_{Tn} in the step S3 are determined by the following method:
performing a first derivative on the curve to obtain a derivative curve of viscosity versus content; when a slope of the derivative curve undergoes an abrupt change as the modifier content increases, the modifier content corresponding the abrupt change is recorded as the transition point.

3. The method for determining the optimal construction temperature of the modified asphalt at the different contents of the modifier as claimed in claim 1, wherein in the step S1, the contents of the modifier vary linearly in a gradient between 1% and 10%.

4. The method for determining the optimal construction temperature of the modified asphalt at the different contents of the modifier as claimed in claim 1, wherein in the step S1, a measurement temperature range for the modified asphalt samples is 80° C. to 210° C.

5. The method for determining the optimal construction temperature of the modified asphalt at the different contents of the modifier as claimed in claim 1, wherein in the step S1, a torque range of a viscometer is controlled between 10% and 98% during viscosity measurement.

6. The method for determining the optimal construction temperature of the modified asphalt at the different contents of the modifier as claimed in claim 1, wherein the modifier is styrene-butadiene-styrene or a mixture containing the styrene-butadiene-styrene.

7. The method for determining the optimal construction temperature of the modified asphalt at the different contents of the modifier as claimed in claim 6, further comprising:
testing a relationship between the modifier content and the optimal construction temperature for modified asphalt with different modifier types according to the step S1 to the step S4, and constructing an optimal construction temperature database for different modifiers; determining the optimal construction temperature of the modified asphalt based on a specific modifier type and a specific modifier content of the modified asphalt during the actual construction.

8. The method for determining the optimal construction temperature of the modified asphalt at the different contents of the modifier as claimed in claim 1, wherein when the transition point cannot be determined based on the curve in the step S2, a viscosity measurement temperature for the modified asphalt samples is lowered until the transition point is determined.
